SEQUENCE LISTING (2 sequences)

SEQ ID NO: 1 - DNA, 6020 bp, Artificial Sequence
  Plasmid
aattcttaagaaggagatatacatatgaacaaaccgcagagctgggaagcgcgtgcggaaacctatagcctgtatggctttaccgatatgccgagcctgcatcagcgtggcaccgttgttgttacccatggcgaaggcccgtatatcgttgatgttaacggccgtcgttatctggatgcgaatagcggcctgtggaatatggttgcgggctttgatcataaaggcctgatcgatgcggcgaaagcgcagtatgaacgttttccgggctatcatgcgttttttggccgtatgagcgatcagaccgtgatgctgagcgaaaaactggttgaagtgagcccgtttgatagcggccgtgtgttttataccaacagcggcagcgaagcgaatgataccatggttaaaatgctgtggtttctgcatgcggcggaaggtaaaccgcagaaacgtaaaattctgacccgctggaacgcgtatcatggcgttaccgcggttagcgcgagcatgaccggcaaaccgtataacagcgtgtttggtctgccgctgccgggttttgttcatctgacctgcccgcattattggcgttatggcgaagaaggcgaaaccgaagaacagtttgttgcgcgtctggcgcgtgaactggaagaaaccattcagcgcgaaggcgcggataccattgcgggtttttttgcggaaccggttatgggtgcgggtggtgttattccgccggcgaaaggttattttcaggcgattctgccgatcctgcgcaaatatgatattccggtgatcagcgatgaagtgatttgcggctttggtcgtaccggtaatacctggggttgcgtgacctatgattttaccccggatgcgatcattagcagcaaaaacctgaccgcgggcttttttccgatgggtgcggttattctgggtccggaactgagcaaacgtctggaaaccgcgattgaagcgatcgaagaatttccgcatggttttaccgcgagcggtcatccggttggttgtgcgattgcgctgaaagcgatcgatgtggttatgaacgaaggcctggcggaaaatgttcgtcgtctggcgccgcgttttgaagaacgcctgaaacatattgcggaacgtccgaacattggcgaatatcgcggcattggttttatgtgggcgctggaagcggttaaagataaagcgagcaaaaccccgtttgatggcaatctgagcgtgagcgaacgtattgcgaatacctgcaccgatctgggtctgatttgtcgtccgctgggtcagagcgttgttctgtgcccgccgtttattctgaccgaagcgcagatggatgaaatgttcgataaactggaaaaagcgctggataaagttttcgcggaagttgcgtaataagcttctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgccggggcacaactcaatttgcgggtactgattaccgcagcaaagaccttaccccgaaaaaatccaggctgctggctgacacgatttctgcggtttatctcgatggctacgagggcagacagtaagtggatttaccataatcccttaattgtacgcaccgctaaaacgcgttcagcgcgatcacggcagcagacaggtaaaaatggcaacaaaccacccgaaaaactgccgcgatcgcgcctgataaattttaaccgtatgaatacctatgcaaccagagggtacaggccacattacccccacttaatccactgaagctgccatttttcatggtttcaccatcccagcgaagggccatccagcgtgcgttcctgtatttccggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcatcgatcaccacaattcagcaaattgtgaacatcatcacgttcatctttccctggttgccaatggcccattttcctgtcagtaacgagaaggtcgcgaattcaggcgctttttagactggtcgtaatgaac

SEQ ID NO: 2 - DNA, 5757 bp, Artificial Sequence
  Plasmid
ccacaattcagcaaattgtgaacatcatcacgttcatctttccctggttgccaatggcccattttcctgtcagtaacgagaaggtcgcgaattcaggcgctttttagactggtcgtagggagaccacaacggtttccctctagaaataattttcttaagaaggagatatacatatgatcataggggttcctaaagagataaaaaacaatgaaaaccgtgtcgcattaacacccgggggcgtttctcagctcatttcaaacggccaccgggtgctggttgaaacaggcgcgggccttggaagcggatttgaaaatgaagcctatgagtcagcaggagcggaaatcattgctgatccgaagcaggtctgggacgccgaaatggtcatgaaagtaaaagaaccgctgccggaagaatatgtttattttcgcaaaggacttgtgctgtttacgtaccttcatttagcagctgagcctgagcttgcacaggccttgaaggataaaggagtaactgccatcgcatatgaaacggtcagtgaaggccggacattgcctcttctgacgccaatgtcagaggttgcgggcagaatggcagcgcaaatcggcgctcaattcttagaaaagcctaaaggcggaaaaggcattctgcttgccggggtgcctggcgtttcccgcggaaaagtaacaattatcggaggaggcgttgtcgggacaaacgcggcgaaaatggctgtcggcctcggtgcagatgtgacgatcattgacttaaacgcagaccgcttgcgccagcttgatgacatcttcggccatcagattaaaacgttaatttctaatccggtcaatattgctgatgctgtggcggaagcggatctcctcatttgcgcggtattaattccgggtgctaaagctccgactcttgtcactgaggaaatggtaaaacaaatgaaacccggttcagttattgttgatgtagcgatcgaccaaggcggcatcgtcgaaactgtcgaccatatcacaacacatgatcagccaacatatgaaaaacacggggttgtgcattatgctgtagcgaacatgccaggcgcagtccctcgtacatcaacaatcgccctgactaacgttactgttccatacgcgctgcaaatcgcgaacaaaggggcagtaaaagcgctcgcagacaatacggcactgagagcgggtttaaacaccgcaaacggacacgtgacctatgaagctgtagcaagagatctaggctatgagtatgttcctgccgagaaagctttacaggatgaatcatctgtggcgggtgcttaatgtacaagtaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgctcgatggctacgagggcagacagtaagtggatttaccataatcccttaattgtacgcaccgctaaaacgcgttcagcgcgatcacggcagcagacaggtaaaaatggcaacaaaccaccctaaaaactgcgcgatcgcgcctgataaattttaaccgtatgaatacctatgcaaccagagggtacaggccacattacccccacttaatccactgaagctgccatttttcatggtttcaccatcccagcgaagggccatgcatgcatcgaaattaatacgacgaaattaatacgactcactatagggcaattgcgatca

(19) United States
(12) Patent Application Publication
(10) Pub. No.: US 2009/0117627 A1
(43) Pub. Date: May 7, 2009

(54) PROCESS FOR PREPARING ENANTIOMERICALLY ENRICHED AMINES

(75) Inventors: Kai DODERER, Rodgau (DE); Wolfgang Wienand, Frankfurt am Main (DE); Harald Groeger, Erlangen (DE); Claudia Rollmann, Alzenau (DE)

Correspondence Address: OBLON, SPIVAK, MCCLELLAND MAIER & NEUSTADT, P.C., 1940 DUKE STREET, ALEXANDRIA, VA, 22314, US

(73) Assignee: EVONIK DEGUSSA GmbH, Essen (DE)

(21) Appl. No.: 12/205,371

(22) Filed: Sep. 5, 2008

Related U.S. Application Data
(60) Provisional application No. 61/074,848, filed on Jun. 23, 2008.

(30) Foreign Application Priority Data
Sep. 7, 2007 (DE) 10 2007 042 600.5

(51) Int. Cl. C12P 13/00; C12N 9/02; C12N 9/10; C12N 5/06

(52) U.S. Cl. 435/128; 435/189; 435/193; 435/325

(57) ABSTRACT

A process for preparing enantiomerically enriched amines by reacting a ketone with ammonia or an ammonium salt and a reducing agent in the presence of a catalytic system comprising the components:
a) an amino acid transaminase,
b) an alpha-amino acid which is a substrate of the amino acid transaminase,
c) an amino acid dehydrogenase suitable for preparing the alpha-amino acid,
d) NAD(P)⁺ and
e) an NAD(P)⁺-reducing enzyme, which reacts NAD(P)⁺ with the reducing agent to give NAD(P)H.
   The process can be carried out with catalytic amounts of alpha-amino acid and NAD(P)⁺, and enables an enantioselective reductive amination of ketones.

### CROSS-REFERENCE TO RELATED APPLICATIONS

This application claims priority to DE 102007042600.5, filed Sep. 7, 2007 and to U.S. Provisional Application 61/074,848, filed Jun. 23, 2008, which are both incorporated by reference in their entireties.

### BACKGROUND OF THE INVENTION

1. Field of the Invention

Process for preparing enantiomerically enriched or pure amines from ketones and ammonia or ammonia salts and a reducing agent using a catalytic system containing enzymes.

2. Description of the Related Art

Enantiomerically pure amines find use as “chiral building blocks” in the preparation of active pharmaceutical and agrochemical ingredients. Prominent examples thereof are rivastigmine, cephalosporin and chiral 1-amino-1-arylalkanes. Individual representatives of these compounds are already being produced in amounts of more than 1000 metric tonnes. For the preparation of active pharmaceutical and agrochemical ingredients, optically pure amines are required, since only either the (R)- or the (S)-enantiomer achieves the desired effect. In some cases, the undesired enantiomer may even give rise to a harmful effect. There is therefore a need for processes for preparing enantiomerically enriched amines.

The most utilized route to enantiomerically enriched or enantiomerically pure amines to date is optical resolution proceeding from amine in racemic form. Conventionally, the optical resolution is effected via diastereomeric salts. To this end, stoichiometric amounts of a chiral carboxylic acid are added to the amine in racemic form and the resulting diastereomeric salts are then separated via fractional crystallization. Thereafter, the chiral carboxylic acid has to be removed again and generally recycled for reasons of cost. The undesired enantiomer either has to be disposed of or converted to the racemate and recycled into the production process. These steps are associated with a considerable level of inconvenience and cost.

An alternative to optical resolution via diastereomeric salts is biocatalytic optical resolution, in which an amine derivative is obtained enantioselectively from a racemic amine by means of an enzyme, or amine is released enantioselectively from a racemic amine derivative. For this purpose, it is possible to use lipases, acylases, proteases and many other hydrolases which are known to be able to split racemates stereospecifically. Such processes are known from Bornscheuer and Kazlauskas, Hydrolases in Organic Synthesis (2005), Wiley-VCH Weinheim, and enzyme catalysis in Organic Synthesis, 2nd Edition (2002), ed. Drauz and Waldmann, Wiley-VCH Weinheim.

In these processes, the use of stoichiometric amounts of chiral auxiliary reagents is dispensed with. However, there remains the disadvantage of a limitation of the theoretical yield to 50% based on the starting material present as the racemate and, if appropriate, the additional working step for the recycling of the undesired enantiomer with the associated costs.

These disadvantages, which apply in principle to all optical resolution strategies, can be avoided by an asymmetric synthesis using prochiral starting compounds. The known asymmetric syntheses using transition metal catalysts, however, often do not achieve the required enantioselectivity. Furthermore, the use of transition metal catalysts can also give rise to contents of transition metals in the resulting product which are undesired for pharmaceutical applications.

A further known enzymatic route to enantiomerically enriched amines is the exchange, catalyzed by transaminases, of keto group and amino group between two substrates (FIG. 4).

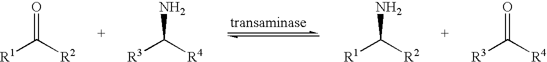

The known transaminases are enantioselective both with regard to the amine group donor used and with regard to the amine formed. Proceeding from prochiral ketones, the reaction therefore forms enantiomerically enriched amines. However, a disadvantage is that the reaction is an equilibrium reaction and therefore generally only a portion of the prochiral ketone used is converted to the desired enantiomerically enriched amine.

Matcham et al., Chimica Oggi 14 (1996) 20-24; Matcham et al., Chimia 53 (1999) 584-589; and WO99/46398 disclose that the equilibrium of the reaction can be shifted to the desired product side when isopropylamine is used as the amine substrate (R³, R⁴=CH₃) and the acetone formed in the reaction is removed by distillation from the reaction mixture. The process can, however, be carried out only with transaminases which accept isopropylamine as an amine group donor. Under the process conditions which are required for the removal of acetone, however, only few transaminases are sufficiently stable.

Shin et al., Biotechnology and Bioengineering 65 (1999) 206-211 and EP 1 818 411 propose shifting the equilibrium through the removal of the ketone formed, by using alanine as the amine substrate and converting the pyruvate formed therefrom further enzymatically with a simultaneously used pyruvate decarboxylase, lactate dehydrogenase or acetolactate synthase. The restriction to alanine as the amine substrate, however, restricts the selection of the transaminases considerably. In order to prepare enantiomerically enriched amines with (R)-configuration, (R)-selective transaminases are required and the amine substrate must likewise be present in the (R)-form. In this process, this requires the use of D-alanine, which is obtainably only with an additional degree of complexity. The alternative is the use of DL-alanine, but L-alanine remains in the reaction mixture in this case and has to be removed and disposed of.

U.S. Pat. No. 3,183,170 describes a process for preparing L-amino acids by reacting an alpha-ketocarboxylic acid with L-glutamic acid in the presence of an L-glutamic acid dehydrogenase, of a hydrogenase, of a transaminase, of a hydrogen acceptor dye, of an electron transport system, of a nitrogen source and of gaseous hydrogen. The process has the disadvantage that the hydrogen acceptor dyes used are strong cell poisons. Moreover, the process is restricted to the amino acid-specific L-glutamic acid dehydrogenase and cannot be employed with amino acid dehydrogenases which tolerate different amino acids as the substrate.

There is therefore a need for a process with which enantiomerically enriched amines can be prepared from ketones and which does not have the disadvantages of the processes known from the background art.

### BRIEF SUMMARY OF THE INVENTION

This object is achieved by the process according to the invention for preparing an enantiomerically enriched amine, in which a ketone is reacted with ammonia or an ammonium salt and a reducing agent in the presence of a catalytic system comprising the following components:
a) an amino acid transaminase,
b) an alpha-amino acid which is a substrate of the amino acid transaminase,
c) an amino acid dehydrogenase suitable for preparing the alpha-amino acid,
d) NAD(P)⁺ and
e) an NAD(P)⁺-reducing enzyme which reacts NAD(P)⁺ with the reducing agent to give NAD(P)H.

Other aspects of the invention are described below.

### BRIEF DESCRIPTION OF THE DRAWINGS

FIG. 1. Yield at maximum conversion of acetophenone to 1-phenylethylamine in the comparative example and in Example 9. The yield and the maximum conversion are 3.5 times higher for the case that the system according to the invention in the application for shifting the equilibrium is applied according to Example 9.

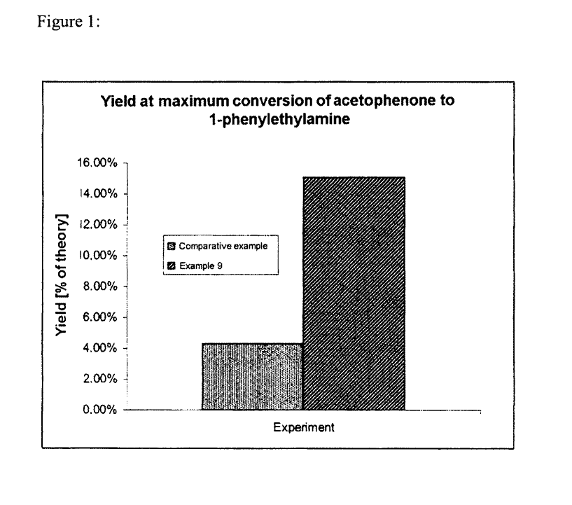

FIG. 2. Restriction map of plasmid pGR15.

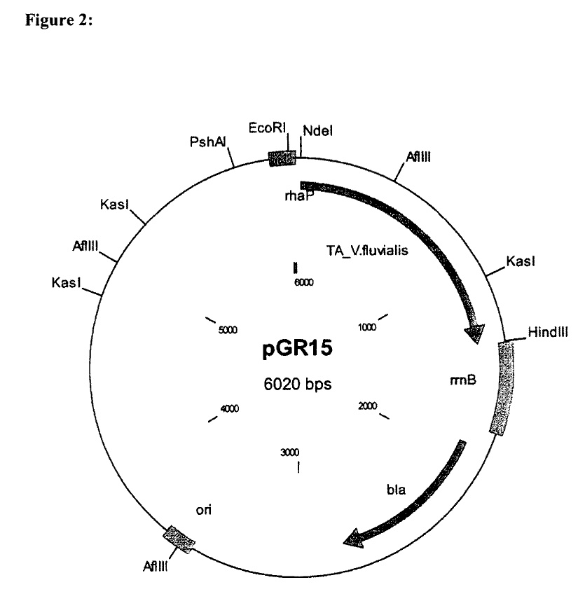

FIG. 3. Restriction map of plasmid pCR4.

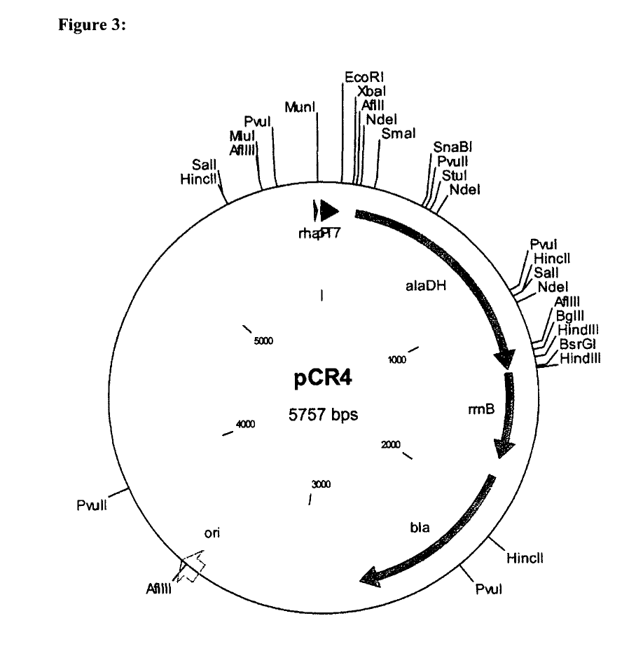

FIG. 4. Enzymatic route to enantiomerically enriched amines by exchange, catalyzed by transaminases, of keto group and amino group between two substrates.

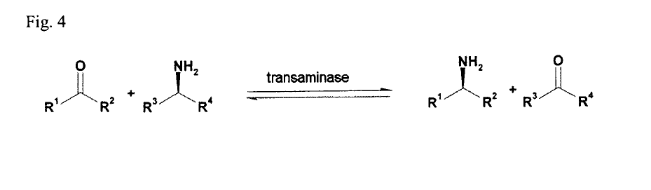

FIG. 5. Reaction scheme depicting the coupling of the three enzymatic reactions to provide a process according to the invention. In this scheme ammonium formate is the reducing agent and ammonium salt, formate dehydrogenase is the NAD⁺-reducing enzyme, AATA denotes an amino acid transaminase, AADH an amino acid dehydrogenase and FDH a formate dehydrogenase.

### DETAILED DESCRIPTION OF THE INVENTION

Amino acid transaminases in the context of the invention are amine group-transferring enzymes of the E.C. 2.6.1.X enzyme class which accept an alpha-amino acid as the substrate.

Amino acid dehydrogenases in the context of the invention are enzymes which react alpha-ketocarboxylic acids with ammonia to give alpha-amino acids and simultaneously oxidize NAD(P)H to NAD(P)⁺.

NAD(P)⁺ represents both nicotinamide adenine dinucleotide (NAD⁺) and salts thereof, and nicotinamide adenine dinucleotide phosphate (NADP⁺) and salts thereof. In the same way, NAD(P)H represents both dihydronicotinamide adenine dinucleotide (NADH) and salts thereof. and dihydronicotinamide adenine dinucleotide phosphate (NADPH) and salts thereof.

The process according to the invention couples three enzymatically catalyzed reactions to give an overall process. In a first reaction catalyzed by an amino acid transaminase, the ketone is reacted with an alpha-amino acid to give the enantiomerically enriched amine and the alpha-ketocarboxylic acid corresponding to the alpha-amino acid. In a second reaction catalyzed by an amino acid dehydrogenase, the alpha-ketocarboxylic acid is reacted with ammonia or an ammonium salt and NAD(P)H to reform the alpha-amino acid and form NAD(P)⁺. In a third reaction catalyzed by an NAD(P)⁺-reducing enzyme, NAD(P)⁺ is reacted with a reducing agent, thus reforming NAD(P)H. The resulting overall reaction is an enantioselective reductive amination of the ketone, in which the alpha-amino acid and NAD(P)⁺ are required only in catalytic amounts and are not consumed for the preparation of the enantiomerically enriched amine.

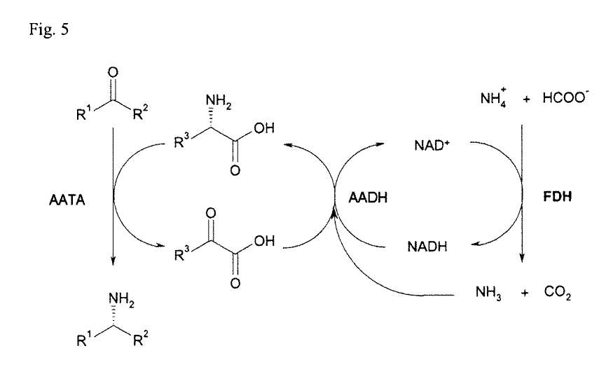

The following formula scheme (FIG. 5) shows the coupling of the three enzymatic reactions to give the process according to the invention using the example of ammonium formate as the reducing agent and ammonium salt, and formate dehydrogenase as the NAD⁺-reducing enzyme. In this scheme, AATA denotes an amino acid transaminase, AADH an amino acid dehydrogenase and FDH a formate dehydrogenase.

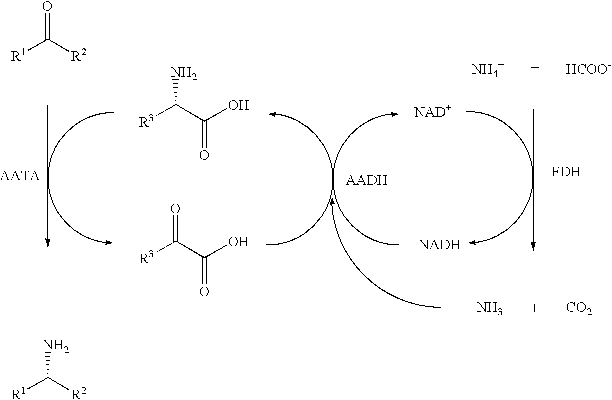

The present invention thus also relates to the use of a catalytic system comprising
a) an amino acid transaminase,
b) an alpha-amino acid which is a substrate of the amino acid transaminase,
c) an amino acid dehydrogenase suitable for preparing the alpha-amino acid,
d) NAD(P)⁺ and
e) an NAD(P)⁺-reducing enzyme which reacts NAD(P)⁺ with the reducing agent to give NAD(P)H for preparing an enantiomerically enriched amine from a ketone.

For the processes according to the invention, it is possible in principle to use all transaminases suitable for the conversion of ketones. Suitable amino acid transaminases are known from Yun et al., Applied and Environmental Microbiology 70 (2004) 2529-2534; Shin et al., Bioscience, Biotechnology, and Biochemistry 65 (2001) 1782-1788; Kaulmann et al., Enzyme and Microbial Technology 41 (2007) 628-637; Shin et al.; Applied Microbiology and Biotechnology 61 (2003) 463-471; Shin et al., Biotechnology and Bioengineering 65 (1999) 206-211; Matcham et al., Chimia 53 (1999) 584-589; and WO 99/46398.

Preference is given to using omega-transaminases which are known from EP 0 404 146. Particularly suitable are omega-transaminases from Vibrio fluvialis, especially Vibrio fluvialis strain JS17; Alcaligenes denitrificans, especially Alcaligenes denitrificans strain Y2K-2; Klebsiella pneumoniae, especially Klebsiella pneumoniae strain YS2F; and Bacillus thuringiensis, especially Bacillus thuringiensis strain JS64.

The alpha-amino acid used may be any alpha-amino acid which is a substrate of the amino acid transaminase. Preference is given to using, as the alpha-amino acid, a proteinogenic amino acid, and it is possible to use both the natural L-amino acids and the D-enantiomers thereof or any desired mixtures of the enantiomers, for example a racemic mixture. Particularly preferred amino acids are leucine, alanine, phenylalanine and glutamic acid.

The alpha-amino acid can be used in a catalytic amount in the process according to the invention. The total amount of alpha-amino acid and of the alpha-keto acid corresponding to the alpha-amino acid, based on the total amount of ketone, is preferably in the range of 1 to 50 mol %, preferably 2 to 10 mol %.

In a preferred embodiment of the process according to the invention, at the start of the reaction, it is not the alpha-amino acid that is initially charged, but rather the alpha-ketocarboxylic acid corresponding to the alpha-amino acid, which has a keto group in place of the amino group. This embodiment is particularly advantageous for the preparation of enantiomerically enriched amines which have to be prepared using a D-amino acid or a non-proteinogenic L-amino acid, since the more readily obtainable alpha-ketocarboxylic acid corresponding to the amino acid can be used for the process in place of an amino acid which is difficult to obtain.

The amino acid dehydrogenase used may be any NAD(P)H cofactor-dependent dehydrogenase with which the alpha-amino acid can be prepared from the alpha-ketocarboxylic acid corresponding to the alpha-amino acid. Suitable amino acid dehydrogenases are known from Oshima et al., International Industrial Biotechnology 9 (1989) 5-11; Ohsima et al., European Journal of Biochemistry 191 (1990) 715-720; Khan et al., Bioscience, Biotechnology and Biochemistry 69 (2005) 1861-1870; Hummel et al., Applied Microbiology and Biotechnology 26 (1987) 409-416 and Bommarius in Enzyme Catalysis in Organic Synthesis, 2nd Edition (2002), ed. Drauz and Waldmann, Wiley-VCH Weinheim.

The amino acid dehydrogenase is preferably a leucine dehydrogenase, an alanine dehydrogenase, a phenylalanine dehydrogenase or a glutamate dehydrogenase. Particularly suitable alanine dehydrogenases are those from Bacillus sphaericus, especially Bacillus sphaericus strain DSM642; glutamate dehydrogenases from Bacillus subtilis, especially Bacillus subtilis strain ISW1214; phenylalanine dehydrogenases from Rhodococcus sp., especially Rhodococcus sp. strain M4, Bacillus sphaericus and Thermoactinomyces intermedius. Particular preference is given to using a leucine dehydrogenase from Bacillus cereus. For the preparation of D-amino acids, preference is given to using a D-amino acid dehydrogenase which is described in Vedha-Peters et al., Journal of the American Chemical Society 128 (2006) 10923-10929 as a mutant of meso-diaminopimelic acid D-dehydrogenase from Corynebacterium glutamicum.

Preference is given to using a combination of an amino acid transaminase and an amino acid dehydrogenase which are balanced to one another in terms of their stereoselectivity for the alpha-amino acid, which means that either the amino acid dehydrogenase converts the alpha-keto acid corresponding to the alpha-amino acid selectively to the S-enantiomer of the alpha-amino acid and the amino acid transaminase reacts the S-enantiomer of the alpha-amino acid selectively with the ketone, or that the amino acid dehydrogenase converts the alpha-keto acid corresponding to the alpha-amino acid selectively to the R-enantiomer of the alpha-amino acid and the amino acid transaminase reacts the R-enantiomer of the alpha-amino acid selectively with the ketone.

In the process according to the invention, in addition, a reducing agent is used in combination with an NAD(P)⁺-reducing enzyme which reacts NAD(P)⁺ with the reducing agent to give NAD(P)H. Suitable NAD(P)⁺-reducing enzymes are known from Enzyme Catalysis in Organic Synthesis, 2nd Edition (2002), ed. Drauz and Waldmann, Wiley-VCH Weinheim.

In a preferred embodiment, the reducing agent used is a salt of formic acid and the NAD(P)⁺-reducing enzyme used is a formate dehydrogenase. The reducing agent is more preferably ammonium formate, which can also be obtained in situ from formic acid and ammonia. Preference is given to using a formate dehydrogenase from Candida boidinii or a mutant derived therefrom. Likewise suitable are the formate dehydrogenases described by Tishkov et al. in Biomolecular Engineering 23 (2006) 89-11. This embodiment has the advantage that the reaction product formed from the reducing agent is only carbon dioxide, which simplifies the workup of the reaction mixture.

In a further preferred embodiment, the reducing agent used is glucose and the NAD(P)⁺-reducing enzyme used is a glucose dehydrogenase. Particularly suitable glucose dehydrogenases are those from Bacillus subtilis, Bacillus megaterium and Thermoplasma acidophilum.

Alternatively, the reducing agent used may be a salt of phosphorous acid and the NAD(P)⁺-reducing enzyme used may be a phosphite dehydrogenase. Suitable phosphite dehydrogenases are known from Relyea et al., Bioorganic Chemistry 33 (2005) 171-189.

Likewise suitable is glucose-6-phosphate as the reducing agent in combination with a glucose-6-phosphate dehydrogenase as the NAD(P)⁺-reducing enzyme.

In the process according to the invention, NAD(P)⁺ and NAD(P)H can be used in catalytic amounts. The total amount of NAD(P)⁺ and NAD(P)H, based on the total amount of ketone, is preferably in the range of 0.001 to 5 mol %, preferably 0.01 to 1 mol %. At the start of the reaction, it is possible to initially charge either NAD(P)⁺ or NAD(P)H as desired.

The enzymes used in the process according to the invention, i.e. the amino acid transaminase, amino acid dehydrogenase and the NAD(P)⁺-reducing enzyme, can be used in the process either dissolved or immobilized on a support. In a preferred embodiment, the enzymes are used in the form of a whole-cell catalyst, i.e. in the form of a cell which expresses all three enzymes. Preference is given to using a recombinant whole-cell catalyst, i.e. a cell which has been genetically modified such that it expresses at least one of the three enzymes from a non-native gene sequence. Particular preference is given to using, as the recombinant whole-cell catalyst, a bacterium, especially an Escherichia coli bacterium, which overexpresses amino acid transaminase, amino acid dehydrogenase and the NAD(P)⁺-reducing enzyme.

In the process according to the invention, the reaction is effected preferably in an aqueous reaction medium. The aqueous phase may additionally comprise a water-miscible solvent, preferably in an amount of 1 to 20% by weight based on the overall reaction mixture. Suitable solvents are alcohols, especially methanol, ethanol and isopropanol, glycols, especially ethylene glycol and propylene glycol, and also tetrahydrofuran, dimethyl sulfoxide and dimethylformamide.

The aqueous reaction medium preferably has a pH in the range of 6 to 9, more preferably 7 to 8. Preference is given to establishing a pH within this range by means of a buffer composed of or comprising a suitable inorganic or organic acid and the ammonium salt thereof. For example, the pH can be established by means of buffers composed of or comprising phosphoric acid and an ammonium phosphate or buffers composed of or comprising a carboxylic acid and the ammonium carboxylate thereof. Alternatively, the pH can also be established by pH-regulated metered addition of ammonia or of an inorganic or organic acid to the reaction medium.

In a preferred embodiment of the process according to the invention, the reaction is effected in a biphasic system composed of or comprising an aqueous phase and an organic phase. The organic phase may consist of the ketone used and/or the amine formed. However, preference is given to forming an organic phase by adding a water-immiscible solvent. Suitable solvents are aliphatic hydrocarbons, especially hexane and heptane, aromatic hydrocarbons, especially toluene and xylenes, dialkyl ethers, especially methyl tert-butyl ether and ethyl tert-butyl ether, and carboxylic esters, especially ethyl acetate. The reaction in a biphasic system also enables, in the case of use of sparingly water-soluble ketones or in the case of preparation of sparingly water-soluble amines, a high volume yield which is not limited by the solubility of ketone or amine. Furthermore, in the case of reaction in a biphasic system, it is also possible to prevent inhibitions of one of the enzymes used by the ketone used or the amine formed. The reaction in a biphasic system also enables simple removal of the amine product from the enzymes used and the recovery thereof by a phase separation, if appropriate after establishing a pH in the range of 9 to 11 beforehand.

The ketones used are preferably dialkyl ketones, alkyl aryl ketones, alkyl heteroaryl ketones and alkyl aralkyl ketones, where the alkyl groups, aryl groups and heteroaryl groups may be substituted by non-enzyme-inhibiting groups. The ketone can be initially charged in the process according to the invention at the start of the reaction. However, preference is given to initially charging only a portion of the ketone to be converted at the start of the reaction and to metering in the remaining portion of the ketone to be converted according to the conversion of ketone. Such a metered addition of the ketone makes it possible to prevent inhibition of the enzymes used by the ketone. Ketones having a melting point of more than 30° C. are preferably used as solutions in a solvent, in which case it is possible to use either the above-described water-miscible solvents or the above-described water-immiscible solvents. The total amount of ketone based on the reaction volume is preferably more than 10 g/l, more preferably more than 50 g/l and especially more than 100 g/l. The upper limit for the total amount of ketone based on the reaction volume is determined by the solubilities of ketone used and amine formed, especially by the solubility of the ammonium salt corresponding to the amine when the reaction is performed at a pH below the pKa of this ammonium salt. The total amount of ketone based on the reaction volume is preferably less than 500 g/l.

The ammonia required in the process according to the invention or the ammonium salt can either be initially charged at the start or be metered in during the reaction according to the conversion of ketone. Preference is given to initially charging ammonia or ammonium salt and if appropriate metering them in such that the total amount of ammonia and ammonium salt is always present in a stoichiometric excess relative to the ketone during the reaction.

The process according to the invention can be performed either batchwise or continuously. In the case of a batchwise reaction regime, the reaction is effected preferably in a stirred tank reactor or in a membrane reactor. In a continuous reaction regime, the reaction is effected preferably in a membrane reactor or with an enzyme immobilized on a solid support in a fixed bed reactor. The reaction in a membrane reactor enables simple retention of the enzymes used in the process according to the invention in the reactor. In the case of use of a recombinant whole-cell catalyst, the enzymes can also be recovered by removing the cells by means of filtration or centrifugation.

As the comparative example shows, the reduction conditions according to Shin and Kim, et al. Biotechnology and Bioengineering 65 (1999), page 206-211, i.e. with a transaminase and an amino acid (alanine), afforded end yields of 4.3% of theory of 1-phenylethylamine from the acetophenone substrate.

Use of an inventive reduction system composed of or comprising an amino acid (alanine), transaminase, amino acid dehydrogenase, NADH, formate dehydrogenase and ammonium formate, in contrast, afforded a 3.5 times higher end yield of 1-phenylethylamine from acetophenone (15.1% of theory according to example 9). This was surprising and unforeseeable and means a great advantage over the background art.

The process according to the invention thus enables the preparation of enantiomerically enriched amines from ketones, without stoichiometric amounts of a chiral assistant being required for this purpose, and has the advantage over the known processes that a suitable combination of alpha-amino acid or alpha-keto acid and amino acid dehydrogenase allows virtually any amino acid transaminase to be used for the conversion of the ketone, such that the amino acid transaminase with which the best enantioselectivity can be achieved in each case can be used for each ketone.

The examples which follow are intended to illustrate the invention, but do not constitute any restriction in the embodiments.

### EXAMPLE 1

### Construction of the Expression Strains

The plasmids pGR15, an E. coli expression plasmid for expressing the transaminase from Vibrio fluvialis under the rhamnose promoter (SEQ ID NO: 1), and pCR4, an E. coli expression plasmid for expressing alanine dehydrogenase from Bacillus subtilis under the rhamnose promoter (SEQ ID NO: 2) were purchased as synthetic constructs from Geneart (Regensburg, Germany).

The plasmids were transformed into E. coli by the customary molecular biology methods (e.g. Sambrook, J., E. F. Fritsch and T. Maniatis (1989). Molecular cloning: a laboratory manual. Cold Spring Harbor, N.Y., Cold Spring Harbor Laboratory), and cultivated on LB agar plates with 100 μg/ml of ampicillin for selection.

### EXAMPLE 2

### Recombinant Expression of the Transaminase from Vibrio fluvialis in E. coli DSM14459

To express the transaminase from Vibrio fluvialis, E. coli DSM14459 (pGR15) was cultivated in 50 ml of LB medium containing 100 μg/ml of ampicillin for plasmid selection. The expression was initiated immediately by adding 2 g/l of L-rhamnose. After incubating at 37° C. while shaking for 24 h, the cells were centrifuged off, resuspended in 10 ml of sodium phosphate buffer (50 mM, pH 7.5) and digested by ultrasound. After centrifuging off the cell fragments, the supernatant was used for further experiments (raw cell extract). The protein content was determined according to Bradford, and the enzyme activity was determined by the transaminase activity test.

### EXAMPLE 3

### Recombinant Expression of the Alanine Dehydrogenase from Bacillus subtilis in E. coli DSM14459

To express the alanine dehydrogenase from Bacillus subtilis, E. coli DSM14459 (pCR4) was cultivated in 50 ml of LB medium containing 100 μg/ml of ampicillin for plasmid selection. The expression was initiated immediately by adding 2 g/l of L-rhamnose. After incubating at 30° C. while shaking for 24 h, the cells were centrifuged off, resuspended in 10 ml of sodium phosphate buffer (50 mM, pH 7.5) and digested by ultrasound. After centrifuging off the cell fragments, the supernatant was used for further experiments (raw cell extract). The protein content was determined according to Bradford and the enzyme activity was determined by the amino acid dehydrogenase activity test.

### EXAMPLE 4

### Gas Chromatography Determination of the 1-phenylethylamine and Acetophenone Concentration

The separation of 1-phenylethylamine and acetophenone was carried out with a WCOT Chrompak 7422 column (length: 25 m, CP-WAX phase). The carrier gas used was helium with a flow rate of 1 ml/min. The separation was effected with a 2-stage temperature gradient, beginning at 50° C. with a heating rate of 0.5° C./min up to 60° C., then with a heating rate of 100° C./min up to 220° C., which was then maintained for 6 min. The detection was effected with a flame ionization detector. The retention time of 1-phenylethylamine was 24.2 min; that for acetophenone was 24.9 min.

### EXAMPLE 5

### Amino Acid Dehydrogenase Activity

The amino acid dehydrogenase activity was determined spectrophotometrically. To this end, the decrease in the NADH concentration was monitored with reference to the decrease in the absorption at a wavelength of 340 nm. In a 0.1 cm quartz glass cuvette, 1 ml of substrate solution containing 1 mmol/l of NADH, 2.5 mmol/l of sodium pyruvate or sodium ketoleucine, 400 mmol/l of ammonium chloride in a potassium phosphate buffer (50 mmol/l, pH 8.2), was mixed with 10 μl of the enzyme sample and the absorption at a wavelength of 340 nm was measured at 30° C. over 10 min. The extinction coefficient ε=6300 l/(mol cm), the path length and the change in the absorption over time were used to calculate the activity.

### EXAMPLE 6

### Formate Dehydrogenase Activity

The formate dehydrogenase activity was determined spectrophotometrically. To this end, the increase in the NADH concentration was monitored with reference to the increase in the absorption at a wavelength of 340 nm. In a 0.1 cm quartz glass cuvette, 1 ml of substrate solution containing 2 mmol/l of NADH, 400 mmol/l of sodium formate in a potassium phosphate buffer (50 mmol/l, pH 8.2), was mixed with 10 μl of the enzyme sample and the absorption was measured at a wavelength of 340 nm at 30° C. over 10 min. The extinction coefficient E=6300 l/(mol cm), the path length and the change in the absorption over time were used to calculate the activity.

### EXAMPLE 7

### Glucose Dehydrogenase Activity

The glucose dehydrogenase activity was determined spectrophotometrically. To this end, the increase in the NADH concentration was monitored with reference to the increase in the absorption at a wavelength of 340 nm. In a 1 cm quartz glass cuvette, 1 ml of substrate solution containing 0.25 mmol/l of NADH, 100 mmol/l of D-glucose in a potassium phosphate buffer (100 mmol/l, pH 7.0), was mixed with 10 μl of the enzyme sample, and the absorption was measured at a wavelength of 340 nm at 30° C. over 10 min. The extinction coefficient ε=6300 l/(mol cm), the path length and the change in absorption over time were used to calculate the activity.

### EXAMPLE 8

### Transaminase Activity

The transaminase activity was determined by gas chromatography. To this end, 1 ml of substrate solution containing 10 mmol/l of sodium pyruvate, 50 mmol/l of 1-phenylethylamine in a potassium phosphate buffer (100 mmol/l, pH 7), was mixed with the enzyme sample and the reaction solution was incubated while shaking at 30° C. At regular intervals, 100 μl of reaction solution were taken as a sample, admixed with 50 μl of 1 M sodium hydroxide solution and extracted with 500 μl of toluene. The organic phase was removed and the content of 1-phenylethylamine and acetophenone was determined by gas chromatography.

### EXAMPLE 9

### With Influence on the Equilibrium Position, According to the Scheme on p. 6 Above and FIG. 5

20 mmol/l of acetophenone, 200 mmol/l of alanine, 90 mmol/l of ammonium formate and 0.3 mmol/l of NADH were mixed in 1 ml of potassium phosphate buffer (100 mmol/l, pH 7), and Vibrio fluvialis transaminase was added up to an end concentration of 20 U/ml, alanine dehydrogenase from Bacillus subtilis up to an end concentration of 400 U/ml, and formate dehydrogenase from Candida boidinii (Jülich, now Codexis) up to an end concentration of 24 U/ml. This solution was incubated at 30° C. while shaking and, after 1, 3, 5, 24 and 48 h, the acetophenone and 1-phenylethylamine content was determined. The maximum conversion of acetophenone was achieved after 48 h.

The yield of 1-phenylethylamine was 8.5% of theory after 24 h and 15.1% of theory after 48 h (in each case 100% selectivity).

### COMPARATIVE EXAMPLE 1

### Without Influence on the Equilibrium Position

30 mmol/l of acetophenone and 300 mmol/l of alanine were mixed in 1 ml of potassium phosphate buffer (100 mmol/l, pH 7), and Vibrio fluvialis transaminase was added up to an end concentration of 20 U/ml. This solution was incubated at 30° C. while shaking and, after 1, 3, 6 and 24 h, the acetophenone and 1-phenylethylamine content was determined. The maximum conversion of acetophenone was achieved after 24 h.

1-phenylethylamine was obtained in a yield of 4.3% of theory (100% selectivity).

Each document, patent, patent application or patent publication cited by or referred to in this disclosure is incorporated by reference in its entirety, especially with respect to the specific material surrounding the citation of the reference in the text. However, no admission is made that any such reference constitutes background art and the right to challenge the accuracy and pertinency of the cited documents is reserved.

1. A process for preparing an enantiomerically enriched amine from a ketone, comprising:
reacting a ketone with ammonia or an ammonium salt and a reducing agent in the presence of a catalytic system comprising:
a) an amino acid transaminase,
b) an alpha-amino acid which is a substrate of the amino acid transaminase,
c) an amino acid dehydrogenase suitable for preparing the alpha-amino acid,
d) NAD(P)⁺ and
e) an NAD(P)⁺-reducing enzyme which reacts NAD(P)⁺ with the reducing agent to give NAD(P)H,
for a time and under conditions producing an enantiomerically enriched amine.

2. The process of claim 1, wherein the amino acid dehydrogenase converts the alpha-keto acid corresponding to the alpha-amino acid selectively to the S enantiomer of the alpha-amino acid, and the amino acid transaminase selectively reacts the S-enantiomer of the alpha-amino acid with the ketone.

3. The process of claim 1, wherein the amino acid dehydrogenase converts the alpha-keto acid corresponding to the alpha-amino acid selectively to the R-enantiomer of the alpha-amino acid, and the amino acid transaminase selectively reacts the R-enantiomer of the alpha-amino acid with the ketone.

4. The process of claim 1, wherein the reducing agent is a salt of formic acid and the NAD(P)⁺-reducing enzyme is a formate dehydrogenase.

5. The process of claim 1, wherein the reducing agent is glucose and the NAD(P)⁺-reducing enzyme is a glucose dehydrogenase.

6. The process of claim 1, wherein the amino acid dehydrogenase is selected from the group consisting of at least one leucine dehydrogenase, alanine dehydrogenase, phenylalanine dehydrogenase, and glutamate dehydrogenase.

7. The process of claim 1, wherein the amino acid transaminase, amino acid dehydrogenase and the NAD(P)⁺-reducing enzyme are used in the form of a recombinant whole-cell catalyst.

8. The process of claim 7, wherein the recombinant whole-cell catalyst is a bacterium which overexpresses amino acid transaminase, amino acid dehydrogenase, and the NAD(P)-reducing enzyme.

9. The process of claim 1, wherein the ketone is selected from the group consisting of at least one dialkyl ketone, alkyl aryl ketone, alkyl heteroaryl ketone, and alkyl aralkyl ketone, where the alkyl groups, aryl groups and heteroaryl groups may be substituted with non-enzyme-inhibiting groups.

10. The process of claim 1, wherein the alpha-keto acid corresponding to the alpha-amino acid is initially charged instead of the alpha-amino acid at the start of the reaction.

11. The process of claim 1, wherein the reaction is effected in an aqueous reaction medium at a pH in the range of 6 to 9.

12. The process of claim 1, wherein the reaction is effected in a biphasic system comprising an aqueous phase and an organic phase.

13. The process of claim 1, wherein only a portion of the ketone to be converted is initially charged at the start of the reaction and the remaining portion of the ketone to be converted is metered in according to the conversion of ketone.

14. The process of claim 1, wherein the total amount of alpha-amino acid and of the alpha-keto acid corresponding to the alpha-amino acid, based on the total amount of ketone, is in the range of 1 to 50 mol %.

15. The process of claim 1, wherein the total amount of alpha-amino acid and of the alpha-keto acid corresponding to the alpha-amino acid, based on the total amount of ketone, is in the range of 2 to 10 mol %.

16. The process of claim 1, wherein the total amount of NAD(P)⁺ and NAD(P)H, based on the total amount of ketone, is in the range of 0.001 to 5 mol %.

17. The process of claim 1, wherein the total amount of NAD(P)⁺ and NAD(P)H, based on the total amount of ketone, is in the range of 0.01 to 1 mol %.

18. A catalytic system comprising:
a) an amino acid transaminase,
b) an alpha-amino acid which is a substrate of the amino acid transaminase,
c) an amino acid dehydrogenase suitable for preparing the alpha-amino acid,
d) NAD(P)⁺ and
e) an NAD(P)⁺-reducing enzyme which reacts NAD(P)⁺ with the reducing agent to give NAD(P)H.

19. The catalytic system of claim 18, further comprising a ketone, ammonia or an ammonium salt, and a reducing agent.

20. The catalytic system of claim 18, which comprises a whole cell catalyst which expresses (a) amino acid transaminase, (c) amino acid dehydrogenase, and (e) a NAD(P)-reducing enzyme.
